(19) United States
(12) Patent Application Publication
(10) Pub. No.: US 2005/0101905 A1
(43) Pub. Date: May 12, 2005

(54) Coding of syringes to monitor their use

(75) Inventor: Alan Forbes Merry, Glendowie (NZ)

Correspondence Address: DARBY & DARBY P.C., P. O. BOX 5257, NEW YORK, NY, 10150-5257, US

(73) Assignee: Safer Sleep Limited

(21) Appl. No.: 10/991,881

(22) Filed: Nov. 17, 2004

Related U.S. Application Data
(63) Continuation of application No. 09/508,499, filed on Apr. 12, 2000.

(30) Foreign Application Priority Data
Sep. 3, 1997 (NZ) 328668

(51) Int. Cl. A61N 1/30

(52) U.S. Cl. 604/19

(57) ABSTRACT

The invention includes a method and apparatus involving the coding, via colours or other identification means, of substances to be used in administration procedure, including providing a support means (10) having areas (11) correspondingly coded (12) so as to provide an additional reference or confirmation means for users of the invention and reducing the inherent risks associated with administration procedures prone to error. The method includes the steps of positioning coded articles in a first position (2a), prior to use, and placing said articles in a second coded position (2b) after use, including the steps of monitoring and recording the use steps in simultaneous actual time, use confirmation monitoring and subsequent use verification or recordal. The invention also includes the step of comparing the procedural administration steps or use of the coded articles against predetermined patterns including audible or other sensory confirmation of predetermined usage or range of usage, and warning against non-predetermined and thus potentially dangerous use.

This invention relates to methods and apparatus for storage, dispensing and use of administrable substances, particularly for anaesthetics. Whilst the invention is primarily directed to anaesthetics, the invention is not limited thereto and may be used in other related areas.

### BACKGROUND TO THE INVENTION

Hitherto, methods and apparatus for storage and use of administrable substances such as anaesthetic drugs and the like, have, in the main, relied upon the skill, alertness and self-imposed systems of practitioners.

It has long been recognised that errors can and do occur, sometimes with disastrous consequences, particularly in the area of anaesthesia where on occasions, owing to tiredness, distraction, adverse conditions (e.g. emergencies) or lack of attention to procedures which have become routine, errors can be made which can result in extremely serious consequences including patient death.

A likelihood of errors is also exacerbated by an increasing complexity of drug administration procedures, types of drugs and their subsets, together with often potentially confusing markings, packaging, concentrations and the like which all but the most alert practitioner might otherwise mistake, especially in emergency or other stressful circumstances.

Many aspects of anaesthesia have highly engineered safety systems, for example, gas bottle pin index systems to prevent the administration of a wrong gas from an anaesthetic machine. Further, gas mixture control systems in place make the administration of a hypoxic gas mixture virtually impossible. These engineering advances operate in conjunction with procedural approaches designed to enhance safety and are backed up by monitors such as in line oxygen monitors and pulse oximeters. In contrast, the administration of intravenous drugs has not changed substantially for many decades, although the number, range and complexity of drugs has undergone an exponential increase.

The flow-on effect is that in some countries practitioners, and organisations such as the hospitals with whom they work often have difficulty in obtaining at reasonable levels an appropriate degree of negligence or malpractice cover, or the costs of dealing with an accident can be astronomical. Further, there is a trend toward the use of criminal law, for example manslaughter prosecutions, in cases of drug administration error which is of concern to those involved in anaesthesia and related activity.

### OBJECTS OF THE INVENTION

It is an object of this invention to come some way in reducing, the likelihood of errors in substance administration, and/or to at least come some way in overcoming the abovementioned problems or at least provide the public with a useful choice.

Other objects of this invention will become apparent from the following description.

### BROAD DESCRIPTION OF THE INVENTION

According to one aspect of this invention there is provided a method of monitoring substance administration including the steps of establishing first and second predetermined coded substance sites for a predetermined coded substance carrier, placing said carrier in an at least partially loaded condition prior to use in said first site and after use in an at least partially discharged condition (relative to said at least partially loaded condition) in said second site and maintaining said carrier in said second site for a predetermined period of time.

According to a further aspect of this invention there is provided a method of monitoring substance administration including the steps of forming a support device having a first predetermined coded substance site for a predetermined coded loaded substance carrier, forming a second predetermined coded site for such carrier, taking said carrier from said first predetermined site for use and, after use, positioning said carrier in the second site.

According to a further aspect of this invention there is provided apparatus for storage and use of at least one administrable substance carrier including a support defining at least one coded site in relation to which the predetermined coded carrier can be positioned, said site coded and adapted to receive and a predeterminedly coded, loaded carrier, said code provided to enable user verification of said carrier relative to said at least one site.

According to a still further aspect of this invention there is provided apparatus for storage and use of at least one administrable substance carrier including a support defining at least one coded site in relation to which the predetermined coded carrier can be positioned, said site provided at least as a set of a first site and a second site, said first site coded and adapted to receive a predeterminedly coded, loaded carrier and a second site at least partially commonly coded and also adapted to receive the carrier, said code provided to enable user verification of said carrier relative to said first and second sites.

According to a still further aspect of this invention there is provided a package of at least one contained administrable substance for administration in accordance with the method as outline above, said package including a support as defined hereinbefore, and wherein at least one of said first sites is charged with a loaded, substantially corresponding coded carrier for said administrable substance and means provided between said carrier and said first coded site for verifying the correct site positioning of said carrier on said site, a second coded site adapted for verification of site position.

Other aspects of this invention will become apparent from the following description. Modifications are envisaged and may be incorporated without departing from the scope or spirit of the invention.

### DESCRIPTION OF THE INVENTION WITH REFERENCE TO THE PREFERRED EMBODIMENTS

The preferred form of the invention will now be described with reference to the accompanying drawings in which:

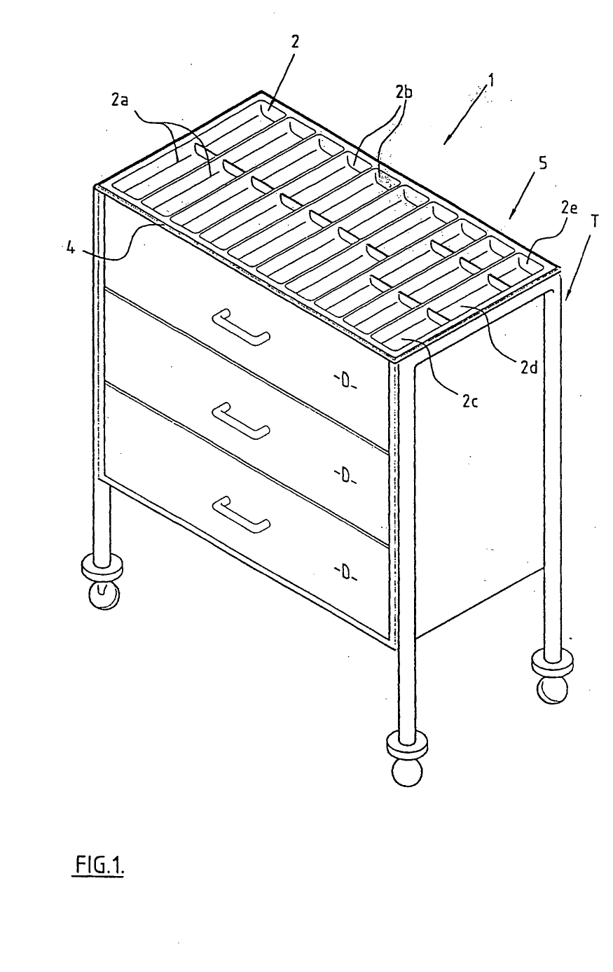

FIG. 1 is a perspective view of an anaesthetic trolley showing the apparatus of the invention mounted therewith.

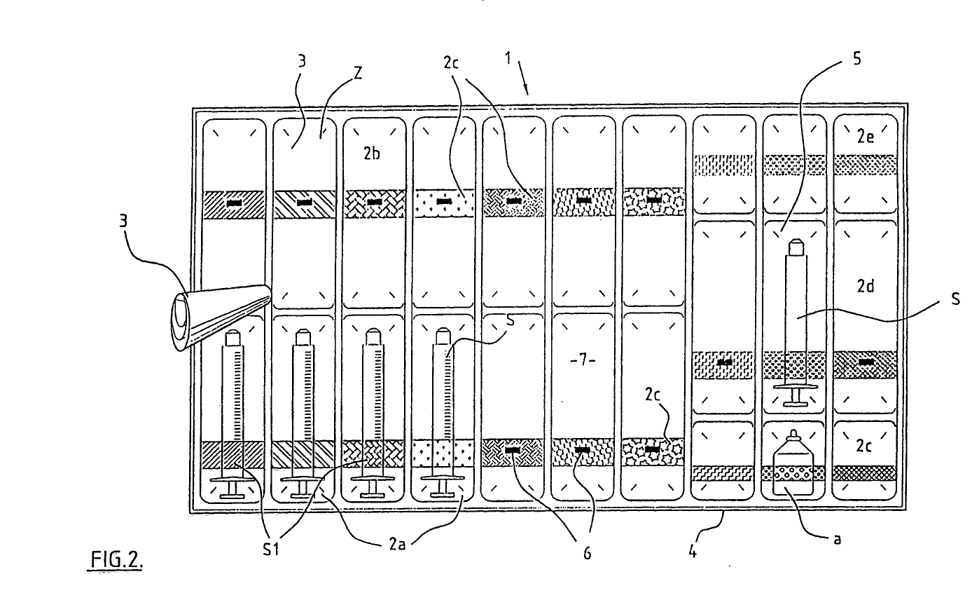

FIG. 2 is an alternative embodiment of the tray according to the invention.

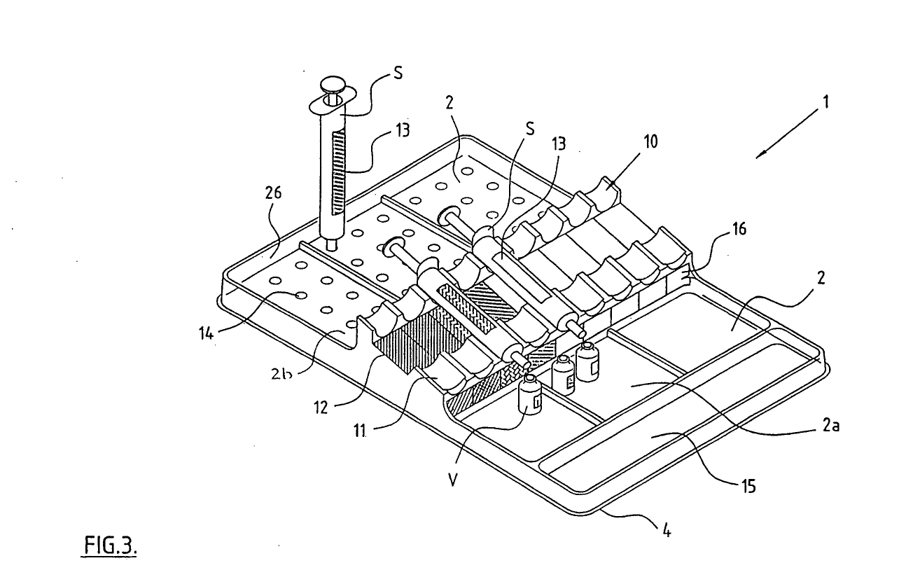

FIG. 3 is an alternative embodiment of the tray according to the invention.

While the preferred embodiment of the invention is described with reference to anaesthesia processes and anaesthetic products and the drawings, the invention is not limited thereto. The invention is applicable in other areas of practice where monitoring of use and a normally predetermined sequence of use is desired.

Many anaesthesia practices are carried out according to relatively standard and repeatable steps, although naturally there often are variations. In other words, there is a sequence through which the practitioner often passes during the course of an operation. For example, the anaesthetist would normally administer drugs or medications in types or classes, amounts (usually volumes) and concentrations dependent on, amongst other things, body mass, degree of anaesthesia required, age, blood pressure, specific patient criteria etc., however, the drugs used in the main generally tend to follow certain predetermined sets of procedures.

There has always previously been a propensity for the practitioner to rely on a combination of skill, experience, memory, colleague verification and verification in relation to notes and procedures to ensure correct drugs are used. The present invention provides a means of reducing reliance on the above procedures to reduce mistakes. In particular, the invention provides a basis for reliance upon sequencing, monitoring and verification, utilising such features as coding, including colour codes, bar codes with comparison against predetermined data and similar techniques and combinations thereof to achieve risk reduction.

With reference to FIG. 1 typically drug ampoules are stored in the drawer D of an anaesthetist's trolley T. There is usually no uniformity of presentation, either visually or spacially and traditionally anaesthetists draw up contents of the ampoules into syringes for administration of the drugs in many steps, all of which are highly error prone.

The present invention provides both a means and apparatus to minimise errors utilising in the preferred form of the invention prefilled colour coded carriers in the form of syringes S (see FIGS. 2 and 3). The syringes S will usually be prefilled by a hospital pharmacy or pharmaceutical manufacturer/supplier and be neatly colour coded by class of drug and other details which may be necessary. Preferably the colours indicate drug classes rather than individual drugs as a drug error between classes is usually much more dangerous than one within a class.

Whilst the preferred form of the invention as described with reference to coding by colours, it is to be appreciated that in alternative forms of the inventions, alternative coding can be incorporated including any one or a combination of:
i. colour coding
ii. colour combinations
iii. pattern codes
iv. numeric codes
v. alpha codes
vi. bar codes

It is however to be appreciate that other forms and combinations of coding may be adopted without departing from the scope or sphere of the invention, as defined in the appended claims.

It will be appreciated that mass production of prefilled syringes and the like is substantially less prone to error than traditional techniques of staff filling to actual demand requirements. Colour coding by class will also minimise the total colours used making the classification system simpler. Whilst colour coding is preferred for classes of drugs, in alternative arrangements it will also be appreciated that a combination of drug class/individual drug may also be provided, for example utilising a two-tier code system or some other detectable identifier or combination of identifier.

Particularly with reference to FIG. 2, in the preferred form coloured syringe S labels S1 are used incorporating the name of the drug in bold print of a size that they will wrap around the syringe S barrel in a way that the colour code can be seen from any likely syringe orientation. In other forms of the invention its is envisaged the syringe body or plunger itself can be colour coded, such as at manufacture.

In the preferred form syringe marking scales will be retained and further, different densities or shades of colour on the label may used to indicate the strength or concentration of the drug.

In alternative arrangements it is envisaged that syringes S or other dispensing apparatus may be prefilled and supplied by drug companies in a substantially complete state. By providing the drugs in a “batch manufactured” manner it is envisaged that further risk reduction will be achieved, the code can also hold this information if required.

In the preferred form of the invention and with reference to FIGS. 1, 2, and 3, syringes S are provided in conjunction with a drug tray 1. It is envisaged that anaesthetic procedures will be divided into preferably three classes according to factors, such as complexity, for example “minor”, “intermediate” and “major”. Sealed sterile plastic trays 1 will be prepackaged with prefilled coded syringes S of the drug classes needed in the “standard” anaesthetic procedure for each of the three classes, resulting in three classes of drug tray 1.

Referring predominantly to FIG. 2 the tray 1 design preferably incorporates separate sites or compartments 2 each, if required, incorporating individually sealed rip-top covers 3 for each compartment 2. Each compartment 2 is the same coded colour 2c as the prefilled syringe S which that compartment 2 is intended to house either by a suitable label or permanent marking 2c on the compartments. The compartments 2 are preferably arranged in a positionally sensitive manner allowing the syringes S to be used from, for example, left to right across the tray 1 as the anaesthetic procedure proceeds.

Each compartment 2 is preferably provided with two subcompartments, a first subcompartment 2a or site, and a second subcompartment 2b or site. The first subcompartment 2a is preferably provided adjacent to a tray front 4 for preloaded, filled syringes S and is designated the “ready” subcompartment 2a. The other, preferably rearward second subcompartments 2b is provided for used or empty syringes S (not shown) and is designated the “used” subcompartment 2b.

In addition to compartments 2a prepacked with filled syringes S, drug trays 1 in each class will also preferably provide also initially empty compartments 2 (including both empty first and second subcompartments 2a and 2b). These empty compartments 2 are provided for use with drugs which are frequently but not always used and are therefore considered not strictly “standard”. The additional compartments 2 can, for example, be supplied with prefilled syringes S from a standard drug drawer D in the anaesthetists trolley T before starting the anaesthetic procedure.

Coding systems and/or labelling will also be used in relation to the additional components 2 by inserting, adhering or otherwise positionally placing both on the syringe S and the additional compartment 2 appropriate codes such as colour codes or other identifier means.

Once a syringe S has been used, if further doses are required these can be obtained by reloading the relevant “ready” subcompartment 2a of the tray 1 with additional prefilled syringes S from a source, perhaps a colour coded drug drawer D elsewhere on the anaesthetists trolley, sympathetically or correspondingly set out and possibly similarly or otherwise coded for ready verification. Used syringes S will accumulate in the relevant “used” subcompartment 2b of the tray 1 as the anaesthetic proceeds and be retained there until the completion of the whole procedure, thus providing ready verification of the identity and amount of drug used at any point in the procedure.

There will always be a certain number of drugs which are not readily available in prefilled syringes S. In most instances, it is envisaged that these drugs will be infrequently used, or are perhaps drugs which are not stable in a plastic syringe S for long periods. A section of the tray 1, for example a righthand section 5 thereof is designed to accommodate drugs only available in ampoules.

In the preferred form of the invention, the coded compartments 2 in this section comprise three subcompartments, a forwardmost compartment 2c for the placement of ampoule A from a colour coded ampoule drawer (not shown) elsewhere in the drug trolley, the middle subcompartment 2e for placement of the syringe S conventionally filled from the ampoule A and colour coded; together with a rearmost compartment 2c for an empty ampoule A (not shown) after the syringe S has been filled.

It will be appreciated that in such a system, keeping track of syringes S and ampoules A until completion of the procedure maintains a visually striking monitor of drug administration and at any time it is possible for practitioners to check at a glance what has been administered and, equally important what has not been, to reduce the potential for error to the individual anaesthetist and also to enhance continuity where one anaesthetist hands over to another during long anaesthetics.

Whilst the invention has been described with reference to a series of “standard” combinations of anaesthetics, it is to be appreciated that alternative arrangements can also provide for the use of, for example, an emergency tray of a generally similar specification to the standard anaesthetics tray 1 prepackaged with prefilled colour codes syringes S of drugs used in an anaesthetic emergency. An emergency drug tray of this type may have the greatest potential to reduce drug error since it is during an emergency that errors are most likely to occur. The emergency tray 1 may be stocked or restocked from an emergency “reserve” drug drawer D in a similar way to the standard trays 1.

The invention envisaged that additional monitoring (including preferably verification, and/or recordal) systems are incorporated into the apparatus. It is envisaged in the preferred form of the invention that each syringe S will incorporate some identification means comparable against predetermined data, for example in a prepared database, to positively identify the contained drug, for example by class, individual drug, concentration and other relevant data to the procedure. Preferably much of such information is incorporated into a conveniently arranged code positioned on the syringe S such as a bar code, however in alternative forms of the invention, other identification means may be provided, for example electronically stored and/or readable identification apparatus, magnetic or digital devices, data information and the like.

In this arrangement, as each syringe S is taken from the ready compartment 2a, it may be, for example, “swiped” under a conveniently positioned reader as part of the drug administration routine the detected code will be compared against the database information and drug identified, whereupon a calm computer generated voice will announce the name and dose of the drug just swiped optionally coupled with a visual display. The response will preferably occur at a time anticipated to be before the actual drug administration. It is envisaged that this will considerably reduce the risk of drug error by supplementing the anaesthetist's already received information with further auditory/visual information to hopefully allow correction of any errors before administration.

In the preferred form of the invention, information received by the monitoring apparatus will be conveyed and stored as a record, for example in a microprocessor based device including a database of drug, drug use and patient information loaded thereon. It is anticipated that the practitioner may, on receiving confirmation of the identity of syringe S from the computerised announcement or verification may physically confirm, for example by depressing a “confirm” key, to confirm verification and/or administration, by taking such action either prior to or subsequent to administration of the identified drug. Measuring apparatus can also optionally be provided connected either directly or indirectly with the syringe S to monitor, measure and record amounts of such drug administration, regardless of the syringe S volume as loaded.

In this way, it will be appreciated that both physical confirmation and verification may be provided, and further, the apparatus will provide a record of the actions of the practitioner. It is envisaged that such a record may be valuable subsequently, should complications arise, or other checking be considered appropriate, and could also be integrated into or connected with known recording apparatus recording general operations monitoring equipment.

The monitoring method and apparatus may incorporate a series “standard” or “specific” administrations previously worked out for the anaesthetic procedure. In such circumstances, it is envisaged that the monitoring apparatus will have such procedures entered into the database and the monitoring apparatus will detect and then compare the removal of syringes S from the “ready” subcompartment 2a of the tray 1 against a predetermined “standard administration order” and not only will provide auditory/visual verification of the syringe S taken, but may also provide an auditory/visual or other warning to the anaesthetist of any variation from the predetermined routine of administration.

Whilst the invention has been described with reference to syringes S and trays 1 with an associated bar code reader, it is envisaged that in an alternative form of the invention the compartments 2 are provided with suitable sensing or detection means 6, for example positioned in the base 7 of each subcompartment 2a/2b. Further, the syringes S are provided with identification means thereon in the form of magnetic/digital devices and others, which can be readily detected by the sensors 6 placed within the base of the tray 1.

The monitoring apparatus is set up to distinguish individual syringes S and drug classes and characteristics in the compartments 2 such that at any stage an accurate and reliable verification of supply, use and countback of drugs/syringes used can be provided and also be monitored against predetermined and anticipated usage manually or via the database as a cross-checking procedure.

Whilst the invention has been described with reference to the provision of sensors 6 placed within the base of the tray 1, in alternative embodiments of the invention, it is envisaged that the upper portion of the trolley T, or some other support apparatus adapted to be used with the tray 1 of this invention may be provided with suitable sensors; the tray 1 being provided of a means substantially inert to interaction between the syringe code and the sensor 6 so as enable simple formation of the trays, or provision of the trays as a liner for separate support apparatus. In this way, it will be appreciated that the cost of tray 1 can be kept to a minimum and further, the sensors/monitoring apparatus will not interfere unduly with necessary sterilisation and other hygiene steps inevitably required.

In the preferred form of the invention, preferably the tray 1 apparatus is provided as a plastics or metal tray 1 able to be sterilised and adapted for ready placement and holding of the syringes S in the required layout for substantially standardised use and providing the first “ready” and the second “used” subcompartments 2a and 2b in a visually separate manner.

In the further embodiment of the invention as described predominantly with reference to FIG. 3, the drug tray 1 is vacuum formed in a thin sheet plastics material, for example transparent or translucent plastics sheet which is capable of being readily cleansed by heat, irradiation and the like. The tray 1 is preferably arranged in a generally “tapered” configuration-so as to be “nestably stackable” with similar trays 1, such that a “pack” of trays 1 can be supplied for general use. Preferably the tray 1 is dimensioned for use with the standard drugs trolley T, substantially as shown in FIG. 1 and further the outer peripheral dimensions of the tray 1 are such that preferably a pair of trays 1 according to FIG. 3 can be mounted side-by-side on the standard drugs trolley T as is typically used in a theatre or other hospital situation, although such use is not essential.

In this form of the invention the sites or compartments 2 are positioned on either side of an enlargement 10 upon which a plurality of arcuate rests or syringe sites 11 are provided. The syringe sites 11 are in this form inclined toward a front 4 of the tray 1 such that syringes S can be readily supported, and viewable by the user. The syringe S after use is able to be positioned in the second compartment 2b which has tapered apertures provided in the second compartment 2b into which a boss B of the syringe S body can optionally frictionally engage, to thus mount the syringe S neatly in a secure and readily visible, verifiable substantially upright manner after use.

The syringe sites 11 also include a predetermined array (preferably three in respect to each compartment 2 “set”) of arcuate rests into which the syringe S can be mounted, inclined forwardly to the user to provide good vision for the user and the syringe S and coding (for example colour coding) at 12 on the sites 11, and on the body of the syringe S.

It will be appreciated that correspondingly coded and possibly prefilled syringes S or dedicated syringes S for particular drugs can be readily positioned on the relevant sites 11 on the rests and on the tray 1 in a verifiable positional relationship.

Preferably a supplementary area 15 is provided across the front 4 of the tray 1 for incidental items and the like as may be required during the course of the anaesthesia operation.

It is envisaged that the enlargement 10 created by the raised area defining the syringe sites 11 will readily enable the enclosed mounting of the monitoring apparatus described hereinbefore, or at least the sensor 6.

It is also envisaged that the drugs trolley T can be arranged on it's upper portion thereof with an enlargement over which the tray 4 can fit. In this assembly coding 12 can be positioned either on the trolley T prior to the application of a tray 1 thereover, where the coding 12 can be “read” through transparent or translucent portions of the tray 1, or alternatively, the coding 12 can be affixed on an underside of the tray 1.

Preferably additional coding 12 may be provided substantially corresponding on a front face 16 of the enlargement 10 to enable additional simple code 12 verification relevant to the particular “row” of the compartments, the syringe sites 11 and in the second compartment 2b.

Where the invention incorporates the use of a “standard” drugs tray 1 incorporating a series of “standard” combinations of anaesthetics, it is to be appreciated that the drugs and drugs tray 1 may be stocked in a “package” form, where a recess provided beneath the enlargement 10 is used for storage of the drugs, syringes S and other items to be used in an anaesthesia operation, optionally contained within a tear-off sheet plastics sheet and the like releasably mounted across adjacent portions of an underside of the tray 1, thus enclosing the items on the underside of the tray 1 which on removal therefrom can be used with the tray 1 in the manner previously described.

The stackable nature of the tray in one alternative embodiment enables a convenient “bulk” store of trays 1 to be held (for example in packs of 10, 20 and the like) for convenient usage when required.

Tray 1 packages can incorporate sets of separate self-adhesive labels or devices holding the codes and for mounting on the tray 1, on syringes S and vials V or ampoules A for matching purposes. The sets of codings may be arranged for either substantially “standard” use codes or alternatively, for special or specific codes to be provided in special use arrangements.

In one alternative form of the invention coded labels arranged for the syringes S are provided in a substantially inverted L shaped configuration, to enable positioning along the syringe body and provision of a readily verifiable code together with a bar code (or interactive indicator for a sensor/monitoring apparatus arrangement) yet still leaving a visual “window” for use of syringe volume graduations thereon.

In further alternative embodiments of the invention, it is envisaged that the additional monitoring checking and notification systems of the apparatus also provide the ability for users to enter further information including, for example specific patient drug allergies and furthermore, to hold on the database or library standard codes and pharmaceutical details for drugs. This facility enables enhancement of the monitoring and in particular, the warning facility described in relation to the preferred embodiment, whereby should a user attempt to give a drug to which a patient is allergic or at variance with predetermined protocols, a timely warning can be given.

In a further embodiment of the invention, the apparatus can verify and record not only drug identity and strength, but also measure the amount of drug actually administered giving the user additional information during the procedure, and also providing a verifiable record subsequently. Furthermore, the code may additionally provide a basis for drug batch identification and to provide raw data and actuation for inventory information, control and drug reordering.

In one embodiment of the invention the monitoring apparatus may be integrated, preferably via a microprocessor to additionally provide an integrated help facility for pharmaceutical information such as dosages, drug properties and the like. One such use would be for the database or library of information on commonly used drugs to be accessible by the user who brings a coded syringe S or other coded drug carrier into proximity with the reader or scanner of the monitoring apparatus and, for example operates a specified key or actuation device to access pharmaceutical information on the drug and its properties during the course of the procedure.

Whilst the invention has been described with reference to a tray 1 and to prefilled syringes S, the invention is not limited to such arrangements and it is envisaged that other drug administration apparatus can be provided and utilised in conjunction with the methods and apparatus described.

Thus, by this invention there is provided a method and apparatus for administration of substances which substantially reduces the risk of errors and provides significant convenience and security.

1. A method of monitoring administration of a therapeutic or anesthetic substance used during a medical or surgical procedure, including the steps of establishing first and second predetermined coded substance sites for predetermined coded substance carrier, wherein the code for the sites and the code for the carrier are independent of each other and the code corresponds to the substance, and wherein the identity of the coded sites once established are fixed for a predetermined period of time of the procedure;
placing the coded carrier in an at least partially loaded condition prior to use of the substance in the first coded site,
placing the coded carrier in the second coded site after use and in an at least partially discharged condition relative to the at least partially loaded condition, and
maintaining the coded carrier in the second coded site for the predetermined period of time of the procedure.

2. A method of monitoring administration of a therapeutic or anesthetic substance during a medical or surgical procedure including the steps of
forming a support device having first and second predetermined coded substance sites for a predetermined coded loaded substance carrier, wherein the code for the site and the code for the carrier are independent of each other and corresponds to the substance, and wherein the identity of the coded sites once established are fixed for a predetermined period of time of the procedure,
taking the carrier from the first predetermined coded site for use and,
after use, positioning the carrier in the second predetermined coded site.

3. A method as claimed in claim 1 including the steps of coding at least portions of said first coded site and second coded site, together with at least a portion of said carrier and verifying use of the substance in said carrier via a predetermined code relationship between said first coded site, said second coded site and said carrier as said carrier is introduced to and removed from said sites.

4. A method as claimed in claim 1 including the step of recording the verification of the carrier at least during a use phase of said carrier.

5. A method as claimed in claim 1 including the step of monitoring movement of said carrier to and from either one or both of predetermined first and second sites.

6. A method as claimed in claim 5 including the step of monitoring said movement via a verification means adapted to detect an encodation of the carrier when said carrier is brought into a predetermined proximity of the verification means.

7. A method as claimed in claim 6 including the step of providing an audible and/or visual signal which is actuated as said carrier is brought into a predetermined proximity with said verification means.

8. A method as claimed in claim 1 including the step of comparing a result of verification of said carrier against predetermined data and including the step of providing a warning when a verification out of range of the predetermined data is detected.

9. A method as claimed in claim 2 including the step of verification by detecting the presence or absence of the carrier in said first and/or said second site.

10. A method as claimed in claim 2 including the step of forming the support to have at least said first and second sites thereon.

11. A method as claimed in claim 1 including the step of verification by bar code scanning.

12. Apparatus for storage and use of at least one administrable substance carrier including a support defining at least one coded site in relation to which the predetermined coded carrier can be positioned, said site coded and adapted to receive and a predeterminedly coded, loaded carrier, said code provided to enable user verification of said carrier relative to said at least one site.

13. Apparatus for storage and use of at least one administrable substance carrier including a support defining at least one coded site in relation to which the predetermined coded carrier can be positioned, said site provided at least as a set of a first site and a second site, said first site coded and adapted to receive a predeterminedly coded, loaded carrier and a second site at least partially commonly coded and also adapted to receive the carrier, said code provided to enable user verification of said carrier relative to said first and second sites.

14. Apparatus as claimed in claim 12 wherein the support defines the first and second sites spaced one from the other but in a related orientation set with at least partially common coding.

15. Apparatus for storage and use of administrable substance carriers as claimed in claim 12, including a verification means adapted to detect an encodation on a carrier when said carrier is brought into a predetermined proximity with said verification means.

16. Apparatus as claimed in claim 15 where said verification means includes a predetermined sequence of encodation detection, and/or a warning device operable to provide a warning when a sequence of encodation out of the predetermined sequence is detected.

17. Apparatus as claimed in claim 15 wherein the verification means is adapted to activate an audible and/or visual or verification of said carrier.

18. Apparatus as claimed in claim 15 wherein the verification means is adapted to detect encodation of a carrier and to actuate a recordal device to provide a record of the encoded carrier brought into predetermined proximity with a detector of said verification means.

19. Apparatus as claimed in claim 15 wherein the verification means detects the presence or absence of a carrier in said first and second sites and is actuated by the introduction and/or removal of said carrier from said sites.

20. Apparatus as claimed in claim 12 wherein said support is formed as a unit defining a recess therebeneath.

21. Apparatus as claimed in claim 20 wherein the support is formed to be nestably stackable over other similar supports.

22. Apparatus as claimed in claim 20, wherein a recess beneath said support is arranged for positioning the verification means therebeneath.

23. Apparatus as claimed in claim 20 wherein the recess beneath said support is arranged for positioning of apparatus for use with the support, a closure portion arranged to be removably secured across at least a portion of a base of said support to enclose said recess.

24. Apparatus as claimed in claim 12 wherein the support is formed at least partially transparent or translucent codes for first coded site and/or second coded site verification visible through said support.

25. Apparatus as claimed in claim 12 wherein the coded first site and/or second sites are arranged with appropriately correspondingly shaped hollow formations to support coded carriers in the form of syringes.

26. Apparatus as claimed in claim 11 wherein the coding used for said first and/or second site, and said carrier is one or all of:
i. a colour code
ii. a colour combination code
iii. a pattern code
iv. a numeric code
v. an alpha code
vi. a bar code.

27. Apparatus as claimed in claim 12 wherein the verification means includes a bar code readably positioned at least on said carrier and a scanner adapted to read said code.

28. A method as claimed in claim 1 including the step of coding said first and second sites and predetermined carrier with a visual code from one or a combination of the following:
i. a colour code
ii. a colour combination code
iii. a pattern code
iv. a numeric code
v. an alpha code
vi. a bar code

29. A package of at least one contained administrable substance for administration in accordance with the method as claimed in claim 1, said package including a support defining at least one coded site in relation to which the predetermined coded carrier can be positioned, said site coded and adapted to receive and a predeterminedly coded, loaded carrier, said code provided to enable user verification of said carrier relative to said at least one site, and wherein at least one of said first sites is charged with a loaded, substantially corresponding coded carrier for said administrable substance and means provided between said carrier and said first coded site for verifying the correct site positioning of said carrier on said site, a second coded site adapted for verification of site position.

30. A package as claimed in claim 29 wherein a releasable restraining means is provided to restrain said loaded carrier with said support until released.

31. A package as claimed in claim 30 wherein said at least one first site is substantially recessed and said restraining means includes a sheet of material at least partially enclosing said loaded carrier in said at least one said first site.

32. A coded syringe for use according to the method as claimed in claim 1.

33. A syringe for use according to the method as claimed in claim 1 encoded with a bar code.

34-38. (canceled)
